(19) United States
(12) Patent Application Publication
(10) Pub. No.: US 2009/0035332 A1
(43) Pub. Date: Feb. 5, 2009

(54) PHARMACEUTICAL FORMULATION

(76) Inventor: Stefan Einar Stefansson, Hafnarfjordur (IS)

Correspondence Address: DUANE MORRIS LLP - NY;PATENT DEPARTMENT, 1540 BROADWAY, NEW YORK, NY, 10036-4086, US

(21) Appl. No.: 12/091,877

(22) PCT Filed: Oct. 27, 2006
(86) PCT No.: PCT/IB2006/003922

§ 371 Date: Sep. 15, 2008

(30) Foreign Application Priority Data
Nov. 3, 2005 (GB) 0522473.8

(51) Int. Cl. A61K 9/00

(52) U.S. Cl. 424/400

(57) ABSTRACT

This invention relates to a stable pharmaceutical formulation containing olanzapine. The composition comprises olanzapine or a pharmaceutically acceptable salt thereof, and one or more suitable pharmaceutical excipients, wherein the composition is coated with a coating comprising polyvinyl alcohol.

This invention relates to a pharmaceutical formulation and in particular to a stable composition for a pharmaceutical dosage form containing olanzapine.

Olanzapine (2-methyl-4-(4-methyl-1-piperazinyl)-10H-thieno[2,3-b][1,5] benzodiazepine) is a psychotropic agent belonging to the class of drugs known as thienobenzodiazepines. Olanzapine is used for the treatment of schizophrenia and is indicated for the prevention of recurrence of manic episodes in patients with bipolar disorder whose manic episode has responded to olanzapine treatment.

U.S. Pat. No. 4,115,568 discloses a general formula of thieno[1,5] benzodiazepines having useful CNS activity. U.S. Pat. No. 5,229,382 discloses olanzapine per se as well its CNS activity.

However, the current commercially available tablets are limited by a high rate of degradation of the drug. Olanzapine is known to suffer from problems associated with the intrinsic nature of olanzapine, e.g. moisture sensitivity, propensity for discoloration, metastability of various crystalline and amorphous forms and degradation after compounding into tablets.

A number of attempts have been made to avoid the degradation of olanzapine. For example, WO 2005/009407 discloses a pharmaceutical composition containing olanzapine particles or powder in which the olanzapine particles or powder have a coating comprising lactose and/or mannitol. This document also discloses a pharmaceutical composition containing olanzapine and suitable excipients in which the olanzapine and excipients have a coating selected from carrageenan, sodium alginate, polyvinyl alcohol-polyethylene glycol graft copolymer, and a titanium dioxide-talc mixture.

There remains, however, a need to provide further and/or improved formulations to avoid degradation and discoloration of olanzapine formulations.

Accordingly, the present invention provides a pharmaceutical composition comprising olanzapine or a pharmaceutically acceptable salt thereof, and one or more suitable pharmaceutical excipients, wherein the composition is coated with a coating comprising polyvinyl alcohol.

It has been found that surprisingly a coating containing polyvinyl alcohol reduces the rate of degradation of olanzapine when compounded into tablets.

The coating of the present invention contains polyvinyl alcohol which is typically partially hydrolysed (e.g. 85-89% hydrolysed). Polyvinyl alcohol is a known excipient, see USP, Ph. Eur. etc., and further explanation is considered unnecessary. Preferably the polyvinyl alcohol consists of vinyl alcohol/acetate monomers only and is not copolymerised with any other monomers. Preferably the polyvinyl alcohol is the sole polymer present in the coating. Preferably the coating further comprises titanium dioxide or talc and particularly preferably a combination of titanium dioxide and talc.

The coating preferably contains a minimum of 20 wt %, more preferably 30 wt % and most preferably 40 wt % of polyvinyl alcohol based on the total weight of the coating. The coating preferably contains a maximum of 80 wt %, more preferably 70 wt %, more preferably 60 wt % and most preferably 50 wt % of polyvinyl alcohol based on the total weight of the coating.

When present, the coating preferably contains a minimum of 10 wt %, more preferably 20 wt % and most preferably 30 wt % of titanium dioxide based on the total weight of the coating. The coating preferably contains a maximum of 60 wt %, more preferably 50 wt %, and most preferably 40 wt % of titanium dioxide based on the total weight of the coating.

When present, the coating preferably contain a minimum of 5 wt %, more preferably 10 wt % and most preferably 20 wt % of talc based on the total weight of the coating. The coating preferably contains a maximum of 50 wt %, more preferably 40 wt %, and most preferably 30 wt % of talc based on the total weight of the coating.

Where the coating contains titanium dioxide and talc the combined total is preferably a minimum of 20 wt %, more preferably 30 wt % and most preferably 40 wt % based on the total weight of the coating. The coating preferably contains a maximum of 70 wt %, more preferably 60 wt %, and most preferably 50 wt % of combined titanium dioxide and talc based on the total weight of the coating. The ratio of titanium dioxide to talc is preferably 90:10 to 10:90, more preferably 70:30 to 50:50 and most preferably about 60:40.

Olanzapine is a known compound and may be synthesised using the procedure disclosed in U.S. Pat. No. 5,229,382.

The coating may be applied using known methods in the art. For example, olanzapine or a pharmaceutically acceptable salt thereof is combined with the appropriate excipients and a suspension, a dispersion or a solution of the coating composition is applied, e.g. by spraying, followed by drying the thus obtained coated composition. The pharmaceutical composition of the present invention is preferably a coated tablet. The polyvinyl alcohol is preferably contained in the coating only.

Olanzapine formulations may be formulated in various dosage forms containing 2 to 20 mg of olanzapine and preferably 2.5, 5, 7.5, 10 or 15 mg of olanzapine.

The formulations contain pharmaceutically acceptable excipients which are well-known in the art. Suitable excipients include binders, disintegrants, fillers and lubricants.

### EXAMPLES

### Preparation Example

A coating composition was prepared containing the following components:

| Component | % | mass/g |
|---|---|---|
| Polyvinyl alcohol | 45.52 | 455.20 |
| Talc | 20.00 | 200.00 |
| Titanium dioxide | 32.00 | 320.00 |
| Xanthan gum | 0.48 | 4.80 |
| Soya lecithin | 2.00 | 20.00 |
| Total | 100.00 | 1000.00 |

### Example 1

| Ingredient | mg per tablet |
|---|---|
| Olanzapine | 10 |
| Lactose anhydrous | 233.2 |
| Microcrystalline Cellulose | 64 |
| Crospovidone | 9.6 |
| Magnesium stearate non bovine | 3.2 |

After compounding, the tablets were coated with the coating composition prepared in Preparation Example at 3.2 mg/tablet.

### Example 2

Tablets coated in accordance with Example 1 were tested for impurities at different temperatures and relative humidity.

% Total Impurities; 10 mg tablets
stored in aluminium/aluminium blisters

| | Tablets from Ex. 1 | Zyprexa* |
|---|---|---|
| 0-month | LOQ** | 0.14 |
| 3-month | LOQ | 0.28 |
| 25° C. 60% RH | | |
| 1-month | 0.05 | 0.21 |
| 40° C. 75% RH | | |

*Commercially available tablet containing olanzapine
**Below limit of quantification

### Example 3

20 mg tablets were compounded with the following composition.

| Ingredient | mg per tablet |
|---|---|
| Olanzapine | 20 |
| Lactose anhydrous | 304.3 |
| Microcrystalline cellulose | 85.3 |
| Crospovidone | 12.8 |
| Magnesium stearate non bovine | 4.3 |
| | 426.7 |

The tablets were kept in an open Petri dish under visible light (VIS) according to ICH guidelines on photostability, over a period of one week. Olanzapine tablets with coating had been coated with coating formulation prepared in the Preparation Example.

% Total impurities in 20 mg tablets

| | |
|---|---|
| Olanzapine | Zyprexa |

| | coated | uncoated | (coated) |
|---|---|---|---|
| 0-month | LOQ | LOQ | 0.07 |
| 1 week VIS | LOQ | 0.17 | 0.13 |

1. A pharmaceutical composition comprising olanzapine or a pharmaceutically acceptable salt thereof, and one or more suitable pharmaceutical excipients, wherein the composition is coated with a coating comprising polyvinyl alcohol.

2. A pharmaceutical composition according to claim 1, wherein the coating further comprises titanium dioxide.

3. A pharmaceutical composition according to claim 1 or 2, wherein the coating further comprises talc.

4. A pharmaceutical composition according to any preceding claim, wherein the coating contains 20 to 80 wt % polyvinyl alcohol based on the total weight of the coating.

5. A pharmaceutical composition according to claims 2 to 4, wherein the coating contains 10 to 60 wt % titanium dioxide based on the total weight of the coating.

6. A pharmaceutical composition according to claims 3 to 5, wherein the coating contains 5 to 50 wt % talc based on the total weight of the coating.

7. A pharmaceutical composition according to any preceding claim, wherein the coating contains 20 to 80 wt % polyvinyl alcohol, 10 to 60 wt % titanium dioxide and 5 to 50 wt % talc based on the total weight of the coating.
